(19) United States
(12) Patent Application Publication
(10) Pub. No.: US 2025/0235274 A1
(43) Pub. Date: Jul. 24, 2025

(54) COMPONENT PRESENCE AND IDENTIFICATION IN SURGICAL ROBOTIC SYSTEM

(71) Applicant: Covidien LP, Mansfield, MA (US)

(72) Inventors: Ranjan Kumar Mishra, Orange, CT (US); Alok Agrawal, New Haven, CT (US); Samuel B. Kesner, Arlington, MA (US); Eric J. Taylor, Southington, CT (US); Michael A. Zemlok, Prospect, CT (US)

(21) Appl. No.: 18/697,829

(22) PCT Filed: Nov. 9, 2022
(86) PCT No.: PCT/IB2022/060796

§ 371 Date: Apr. 2, 2024

Related U.S. Application Data
(60) Provisional application No. 63/389,570, filed on Jul. 15, 2022.
(60) Provisional application No. 63/277,626, filed on Nov. 10, 2021.

(51) Int. Cl. A61B 34/30; A61B 46/10; A61B 90/00; A61B 90/98

(52) U.S. Cl. CPC A61B 34/30; A61B 46/10; A61B 90/08; A61B 90/98; A61B 2034/305; A61B 2090/0808

(57) ABSTRACT

A wireless communication interface is included in a surgical robotic arm and is used to detect various components of the robotic arm, including, an instrument drive unit, a sterile interface module (e.g., adapter), an instrument, an access port, etc. Detection of the components is used to confirm that each of the components are properly assembled by splitting up tag assemblies between multiple components, such that when the components are properly assembled, so are the tag assemblies, allowing for interrogation thereof by the wireless communication interface.

### BACKGROUND

Surgical robotic systems may include a surgical console controlling one or more surgical robotic arms, each having a surgical instrument having an end effector (e.g., forceps or grasping instrument). In operation, one or more robotic arms are moved to a position over a patient and the surgical instrument is guided into a small incision via a surgical access port or a natural orifice of a patient to position the end effector at a work site within the patient's body.

Various components of surgical system and medical devices are identified using electronic tracking, e.g., radio frequency identification (RFID). However, such methods are prone to signal integrity issues when subjected to different sterilization routines and external mechanical loading conditions. Furthermore, current RFID trackers are not configurable and cannot detect multiple components, identify component types, and provide status updates for correct placement as well as continuous monitoring of components.

### SUMMARY

This disclosure provides a wireless communication interface including a reader configured for radio-frequency communication, e.g., RFID, Bluetooth, near field communication (NFC) protocols, to detect and read identification tags associated with multiple components of a surgical robotic arm, including an instrument drive unit (IDU), a sterile interface module (SIM), an instrument, an access port, sterile drapes, stapler reloads, instrument adapters, and the like. The tags may include an antenna and a chip storing identification data pertaining to each of the components. The tags are also configured to communicate with various sensors of the components. The sensors may be configured to measure operation of the components, including, torque, force, proximity, temperature, etc. The tags may be over-molded or embedded on to the components. The tags may be either passive, active (i.e., powered), or may have energy harvesting circuitry.

Each component also includes mechanical interlocking features. Thus, when the components are assembled and are coupled to each, the tags are positioned within a corresponding reading zone of the reader disposed on the robotic arm. This ensures correct component placement including sequence of coupling of each of the components. In addition, the components may have redundant electrical contacts interconnecting each of the components and the tags.

The wireless communication interface is configured to detect all tags within each of the reading zones and may be associated with the respective reader on specific components. The wireless communication interface also supports simultaneous detection of multiple components to guide component placement and assembly in a desired sequence, e.g., attach SIM to IDU, attach instrument to SIM, etc. In addition, to the wireless communication interface, the system is also configured to support wireless data streaming from various sensors of the components, such as torque, force, proximity, temperature etc. Detection of multiple components by multiple readers due to proximity may also be used to evaluate collision boundary of the robotic arm. The orientation of instrument roll can also be identified to support roll calibration of the instrument.

According to one embodiment of the present disclosure, a surgical robotic system is disclosed. The surgical robotic arm includes a robotic joint; a first component configured to couple to the robotic joint; and a second component configured to couple to the first component. The surgical robotic arm also includes a first tag assembly having a first portion disposed in the first component and a second portion disposed in the second component, where the first portion of the first tag assembly and the second portion of the first tag assembly are in electrical communication once the first component is mechanically engaged with the second component; and a wireless communication interface configured to interrogate the first tag assembly.

Implementations of the above embodiment may include one or more of the following features. According to one aspect of the above embodiment, the first component may be an instrument drive unit. The second component may be a sterile interface module configured to secure a sterile drape to the instrument drive unit. The first portion of the first tag assembly may include a first pair of contacts and the second portion of the first tag assembly may include a second pair of contacts, The first pair of contacts and the second pair of contacts are configured to electrically couple to each other once the first component is mechanically engaged with the second component. The first tag assembly may include a first tag antenna. The first portion of the second tag assembly and the second portion of the second tag assembly are in electrical communication once the third component is mechanically engaged with the second component. The wireless communication interface is configured to interrogate the second tag assembly. The first portion of the second tag assembly may include a first pair of contacts, and the second portion of the second tag assembly may include a second pair of contacts, the first pair of contacts and the second pair of contacts of the second tag assembly are configured to electrically couple to each other once the third component is mechanically engaged with the second component. The second tag assembly may include a second tag antenna.

According to another embodiment of the present disclosure, a surgical robotic system is disclosed. The surgical robotic arm includes a robotic joint; an instrument drive unit configured to couple to the robotic joint; and a sterile interface module configured to couple to the instrument drive unit. The surgical robotic arm also includes a first tag assembly may include a first portion disposed in the instrument drive unit and a second portion disposed in the sterile interface module, where the first portion of the first tag assembly and the second portion of the first tag assembly are in electrical communication once the instrument drive unit is mechanically engaged with the sterile interface module; and a wireless communication interface configured to interrogate the first tag assembly.

Implementations of the above embodiment may include one or more of the following features. According to one aspect of the above embodiment, the surgical robotic arm may also include a latch port configured to couple to an access port. The wireless communication interface may include a first antenna disposed along at least a portion of the robotic joint and a second antenna disposed at the latch port. The instrument drive unit is configured to move longitudinally along the robotic joint. The first portion of the first tag assembly may include a first pair of contacts, and the second portion of the first tag assembly may include a second pair of contacts. The first pair of contacts and the second pair of contacts are configured to electrically couple to each other once the instrument drive unit is mechanically engaged with the sterile interface module. The first tag assembly may include a first tag antenna. The first portion of the second tag assembly and the second portion of the second tag assembly are in electrical communication once the instrument is mechanically engaged with the sterile interface module. The wireless communication interface is configured to interrogate the second tag assembly. The first portion of the second tag assembly may include a first pair of contacts, and the second portion of the second tag assembly may include a second pair of contacts. The first pair of contacts and the second pair of contacts of the second tag assembly are configured to electrically couple to each other once the instrument is mechanically engaged with the sterile interface module. The second tag assembly may include a second tag antenna.

According to a further embodiment of the present disclosure, a method for detecting components of a surgical robotic arm is disclosed. The method includes interrogating a first tag assembly having a first portion disposed in a first component and a second portion disposed in a second component. The first portion of the first tag assembly and the second portion of the first tag assembly are in electrical communication once the first component is mechanically engaged with the second component and at least one of the first component and the second component is coupled to a robotic joint. The method also includes determining whether the first component and the second component are mechanically engaged to each other based on the interrogation.

Implementations of the above embodiment may include one or more of the following features. According to one aspect of the above embodiment, interrogating may be performed using a wireless communication interface disposed in the robotic joint. The first portion of the second tag assembly and the second portion of the second tag assembly are in electrical communication once the third component is mechanically engaged with the second component.

According to yet another embodiment of the present disclosure, a surgical robotic arm is disclosed. The surgical robotic arm includes a robotic joint and an instrument drive unit configured to couple to the robotic joint. The surgical robotic arm also includes a plurality of RFID tags disposed on the instrument drive unit and a wireless communication interface configured to output radio signals in a reading zone and to interrogate at least one RFID tag of the plurality of RFID tags.

Implementations of the above embodiment may include one or more of the following features. According to one aspect of the above embodiment, the instrument drive unit is rotatable about a longitudinal axis relative to the robotic joint. The wireless communication interface may also include a longitudinal antenna disposed in parallel to the longitudinal axis. The plurality of RFID tags may be spaced apart in equal angular increments. The instrument drive unit may be configured to move along the longitudinal axis robotic joint relative to the robotic joint. The surgical robotic arm may further include an integrated data chip coupled to the plurality of RFID tags. The integrated data chip may be a storage device and/or a sensor.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a side, schematic view of a holder of the surgical robotic arm with a wireless communication interface according to an embodiment of the present disclosure;

FIG. 6 is a perspective, front view of the holder of the surgical robotic arm with a wireless communication interface according to an embodiment of the present disclosure;

FIG. 7 is a perspective, side view of an instrument drive unit according to an embodiment of the present disclosure;

FIG. 8 is a front view of the instrument drive unit of FIG. 7;

FIG. 9 is a perspective view of a distal portion of the instrument drive unit of FIG. 7;

FIG. 10 is a perspective view of a sterile interface module according to an embodiment of the present disclosure;

FIG. 11 is a front view of the sterile interface module according to an embodiment of the present disclosure;

FIG. 12 is a perspective, top view of an instrument according to an embodiment of the present disclosure;

FIG. 13 is a rear view of the instrument according to an embodiment of the present disclosure;

FIG. 14 is a side view of the instrument, the sterile interface module, and the instrument drive unit in an assembled configuration according to an embodiment of the present disclosure;

FIG. 15 is a perspective view of an access port according to an embodiment of the present disclosure;

FIG. 16 is a flow chart of a method according to an embodiment of the present disclosure; and

FIG. 17 is a front view of the instrument drive unit of FIG. 7 according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “distal” refers to the portion of the surgical robotic system and/or the surgical instrument coupled thereto that is closer to the patient, while the term “proximal” refers to the portion that is farther from the patient.

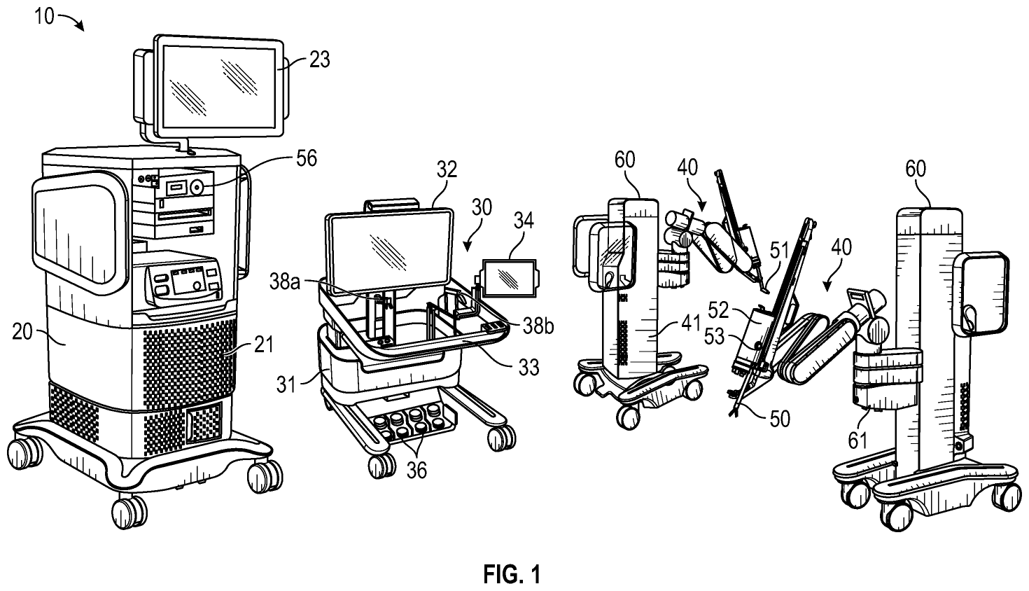

With reference to FIG. 1, a surgical robotic system 10 includes a control tower 20, which is connected to all of the components of the surgical robotic system 10 including a surgical console 30 and one or more movable carts 60. Each of the movable carts 60 includes a robotic arm 40 having a surgical instrument 50 removably coupled thereto. The robotic arms 40 is also coupled to the movable cart 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the present disclosure and output the processed video stream.

The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

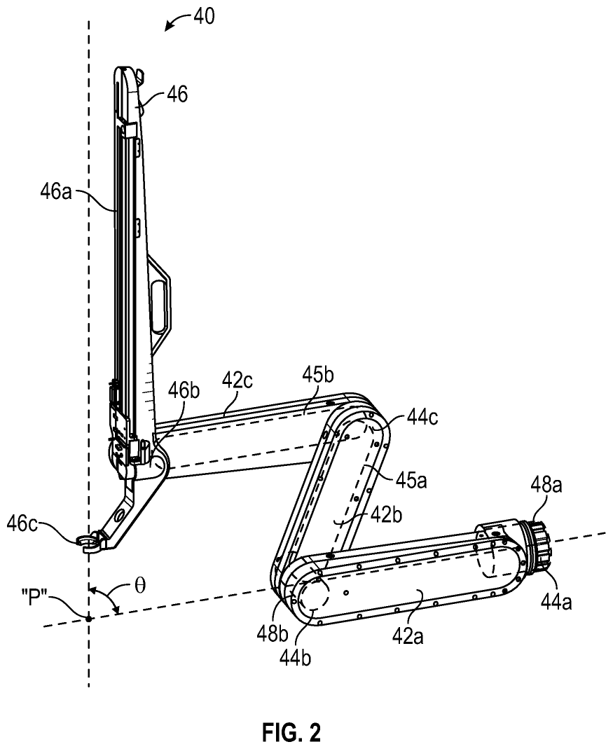

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis.

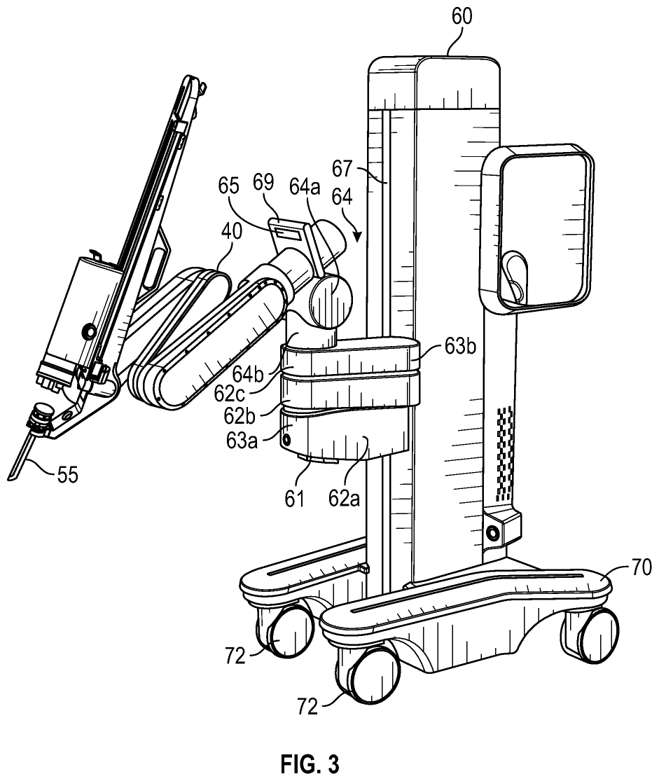

With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints. The mobile cart 60 includes a base 70 having a plurality of wheels 72 allowing for movement of the mobile cart 60. The lift 67 extends vertically from the base 70.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

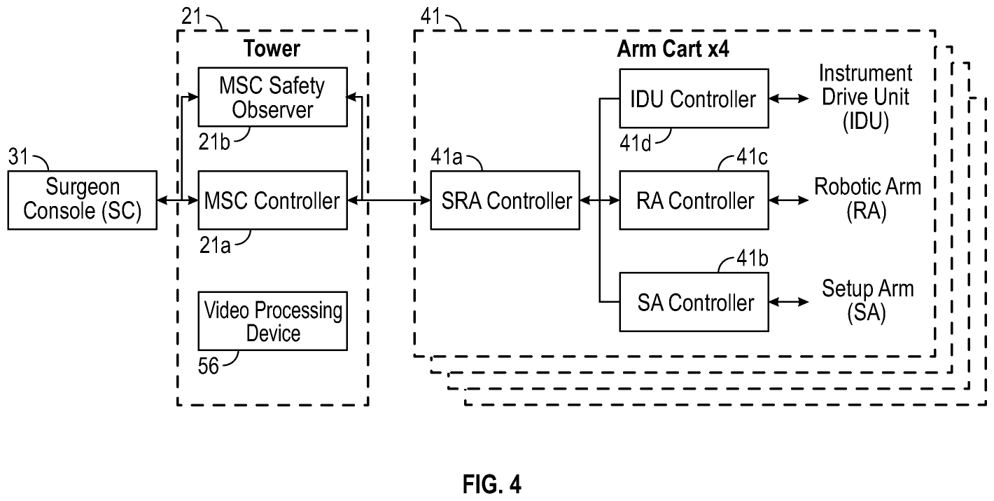

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 61 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (RPY) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

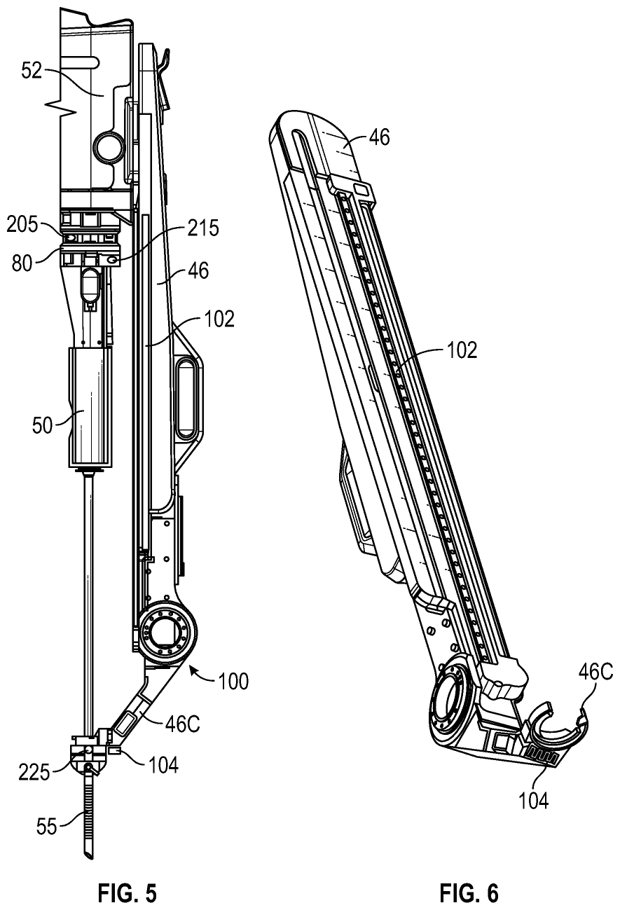

With reference to FIGS. 5 and 6 the holder 46 includes a wireless communication interface 100 having a first antenna 102 and a second antenna 104. The first antenna 102 has a longitudinal shape (e.g., a strip) and is disposed along the second longitudinal axis of the holder 46. The longitudinal shape of the first antenna 102 allows for receiving radio signals along the length of the holder 46, including interrogating of the IDU 52, a sterile interface module (SIM) 80, and the instrument 50. The second antenna 104 is disposed at the port latch 46c. The second antenna 104 is configured to interrogate the access port 55. The antennas 102 and 104 may be embedded into or over-molded to holder 46 and the port latch 46c, respectively. The antennas 102 and 104 may have any suitable shape, e.g., square wave antenna. In addition, the antennas 102 and 104 are coupled to communication circuitry (not shown), e.g., transceiver, configured to transmit and receive wireless signals. The communication circuitry is coupled to the main cart controller 41a, allowing the main cart controller 41a to interrogate components using the antennas 102 and 104. In embodiments, any other controller of the system 10 may be used to control the wireless communication interface 100.

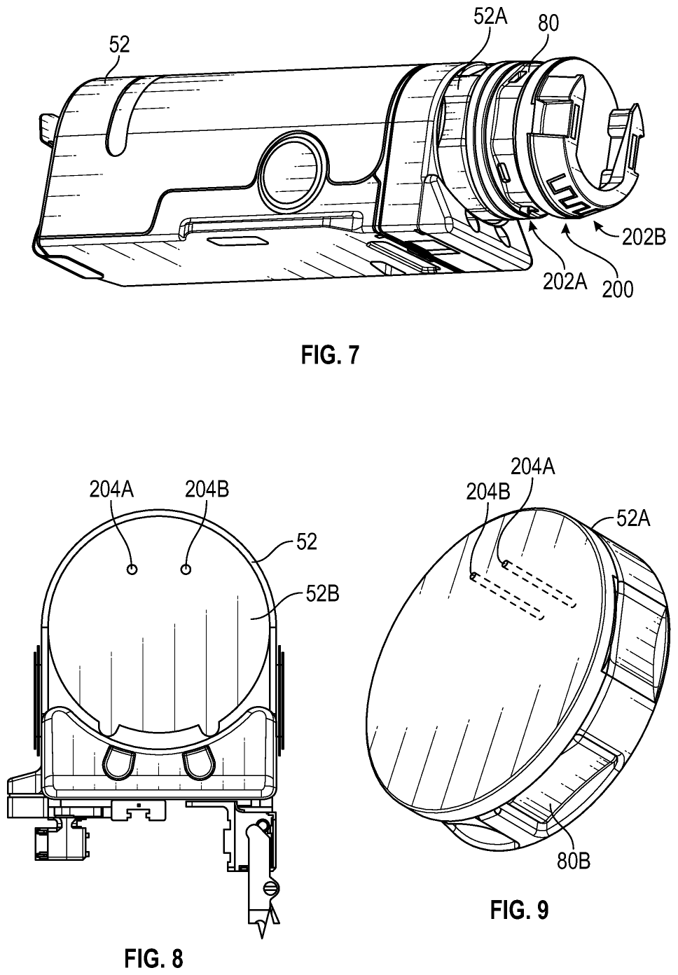

During use, the robotic arm 40 is attached to the access port 55 that is inserted into the patient by attaching the port latch 46c to the access port 55. The IDU 52 is attached to the holder 46, followed by the SIM 80 being attached to a distal portion 52a of the IDU 52 (FIG. 7). Thereafter, the instrument 50 is attached to the SIM 80. The instrument 50 is then inserted through the access port 55 by moving the IDU 52 along the holder 46. The sim 80 includes a plurality of drive shafts configured to transmit rotation of individual motors of the IDU 52 to the instrument 50 thereby actuating the instrument 50. In addition, the SIM 80 provides a sterile barrier between the instrument 50 and the other components of robotic arm 40, including the IDU 52. The SIM 80 is also configured to secure a sterile drape (not shown) to the IDU 52.

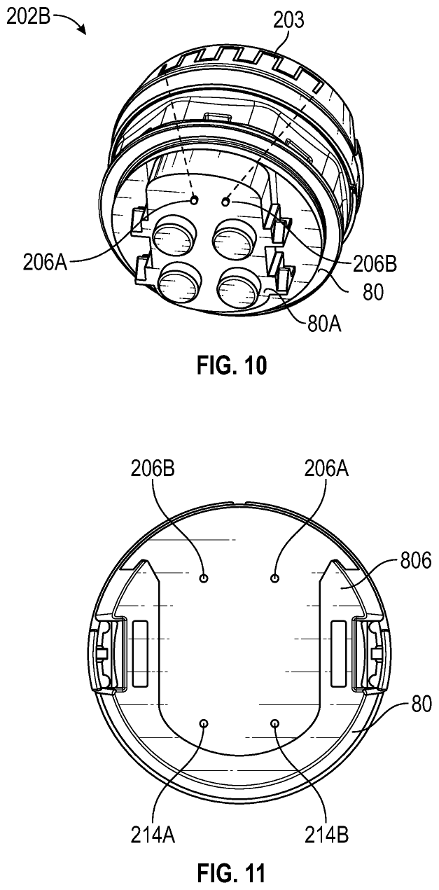

With reference to FIGS. 7-10, the wireless communication interface 100 is configured to interrogate one or more tag assemblies 200, each of which includes first and second portions 202a and 202b, each of which is disposed within their corresponding components. Thus, only after the two portions 202a and 202b are in electrical communication with each other, the tag assembly 200 readable by the wireless communication interface 100, which provides a verification that the two components have been properly assembled.

The first portion 202a of the first tag assembly 200 is disposed in the IDU 52 and the second portion 202b disposed within the SIM 80. The first portion 202a includes a first pair of contacts 204a and 204b that are electrically coupled to each other. The second portion 202b includes a second pair of contacts 206a and 206b that are configured to electrically couple to the first pair of contacts 204a and 204b once the SIM 80 is engaged with the distal portion 52a of the IDU 52. The distal portion 52a and the SIM 80 may include two interlocking mechanical features 52b and 80a, respectively, which may be of any suitable shape, e.g., U-shape, bayonet connection, etc.

In embodiments, a latching mechanism 80b may also be used to provide for a secure, lockable mechanical connection. Once the SIM 80 is mechanically engaged with the distal portion 52a, the first and second portions 202a and 202b are electrically coupled to each other due to alignment of the first and second pairs of contacts 204a, 204b and 206a, 206b. The second portion 202b also includes a tag antenna 203, which may have any suitable shape and configuration. The antenna 203 is functional (i.e., electrical circuit is closed/complete) once the first and second pairs of contacts 204a, 204b and 206a, 206b are electrically coupled to each other, this allows the antenna 203 to be interrogated by the first antenna 102. Thus, once the antenna 203 is responsive to interrogation by the first antenna 102. Thus, interrogation of the antenna 203, which provides verification that the SIM 80 is coupled to the IDU 52.

The second portion 202b may be also coupled to an identification device 205 (FIG. 5) disposed within the SIM 80, which may include any suitable storage device, e.g., flash memory, storing any data (i.e., SIM data) pertaining to the SIM 80, e.g., identification number, model, prior number of uses, maximum number of uses, number of sterilizations, sterilization status, etc.

With reference to FIGS. 11-14, a second tag assembly 210 is shown, with a first portion 212a disposed in the SIM 80 and a second portion 212b disposed within the instrument 50. The first portion 212a includes a first pair of contacts 214a and 214b that are electrically coupled to each other. The second portion 212b includes a second pair of contacts 216a and 216b that are configured to electrically couple to the first pair of contacts 214a and 214b once the SIM 80 is engaged with a proximal portion 50a of the instrument 50. The proximal portion 50a and the SIM 80 include two interlocking mechanical features 50b and 80b, respectively, which may be of any suitable shape, e.g., U-shape, bayonet connection, etc.

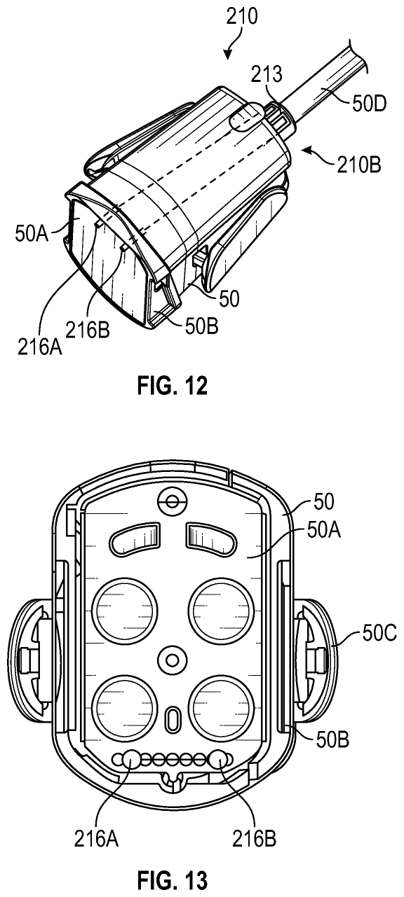

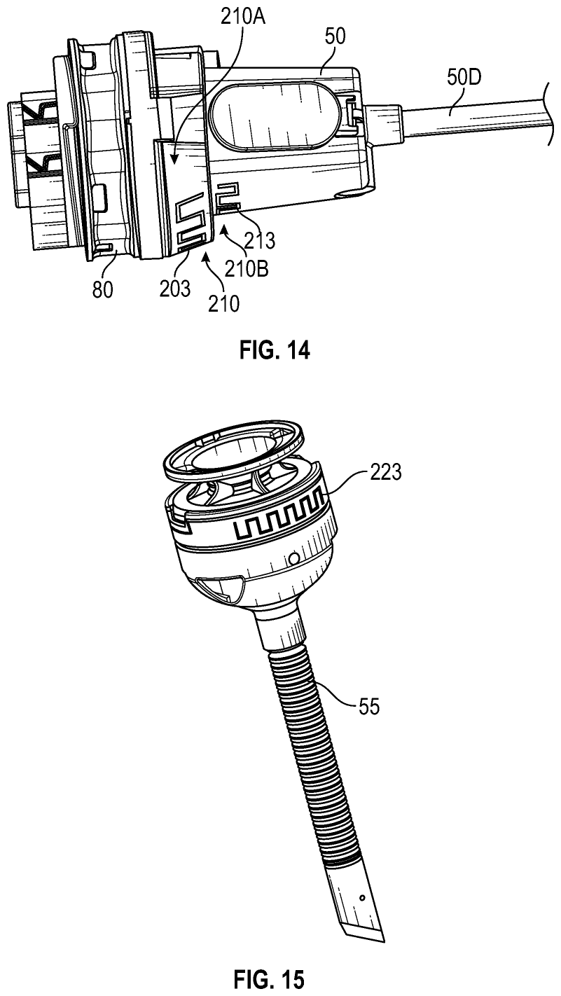

In embodiments, a latching mechanism 50c may also be used to provide for a secure, lockable mechanical connection. Once the SIM 80 is mechanically engaged with the proximal portion 50a, the first and second portions 212a and 212b are electrically coupled to each other due to alignment of the first and second pairs of contacts 214a, 214b and 216a, 216b. The second portion 212b also includes a tag antenna 213, which may have any suitable shape and configuration. The antenna 213 may be disposed at any portion of the instrument 50, e.g., its longitudinal shaft 50d (FIG. 12), proximal housing (FIG. 14), etc. The antenna 213 is functional (i.e., has a complete electrical circuit) once the first and second pairs of contacts 214a, 214b and 216a, 216b are electrically coupled to each other. This allows the antenna 213 to be interrogated by the first antenna 102, which provides verification that the SIM 80 is coupled to the IDU 52.

When disposed on the longitudinal shaft 50d, the antenna 213 may be used for roll calibration of the instrument 50 (i.e., zeroing of rotation angle of the longitudinal shaft 50d) by placing the antenna 213 at a zero location and measuring signal strength, which is based on the distance between the first antenna 102 and the antenna 213.

The second portion 212b may be also coupled to an identification device 215 (FIG. 5) disposed within the instrument 50, which may include any suitable storage device, e.g., flash memory, storing any data (i.e., instrument 50) pertaining to the instrument 50, e.g., identification number, model, prior number of uses, maximum number of uses, sterilization status, etc. The second portion 212b may also be coupled to various sensors, e.g., torque sensors, encoders, thermocouples, etc. disposed within the instrument 50 allowing for transmission of sensor data through the wireless communication interface 100 to the main cart controller 41a.

In embodiments, a single tag assembly may be used having a plurality of portions, each of which is disposed in the IDU 52, the SIM 80, and the instrument 50, which once mechanically engaged, complete the electrical circuit of an antenna, allowing for interrogation by the wireless communication interface 100.

With reference to FIGS. 5 and 15, the access port 55 includes an antenna 223, which is configured to be interrogated by the second antenna 104 of the wireless communication interface 100. During use, the access port 55 is rotated to align the antenna 223 with the second antenna 104 to establish wireless communication therebetween. The antenna 223 may also be coupled to an identification device 225 (FIG. 5) disposed within the access port 55, which may include any suitable storage device, e.g., flash memory, storing any data (i.e., access port 55) pertaining to the access port 55, e.g., identification number, model, prior number of uses, maximum number of uses, sterilization status, etc. The antenna 223 may also be coupled to various sensors, e.g., force sensors, strain gauges, etc. disposed within access port 55 allowing for transmission of sensor data through the wireless communication interface 100 to the main cart controller 41a. Thus, in unassembled state the antennas 213 and 223 are in open circuit and are not readable, and once assembled, the electrical circuit is closed, and the antennas 213 and 223 are readable.

The tag assemblies 200 and 210 may encased in epoxy or any other autoclavable material, such as FR4 (e.g., glass-reinforced epoxy laminate material), that do not decompose or deteriorate when exposed to high levels of heat and moisture. Exposed plastic components of the tag assemblies 200 and 210 may be over-molded. For high number of usage components, such as the SIM 80, over-molded areas may have additional cavities to hold a layer of epoxy to provide additional protections for autoclave exposures. For metal components such as antennas 203, 213, 223, may be soldered onto a pre-defined region that can withstand high number of autoclaves exposures.

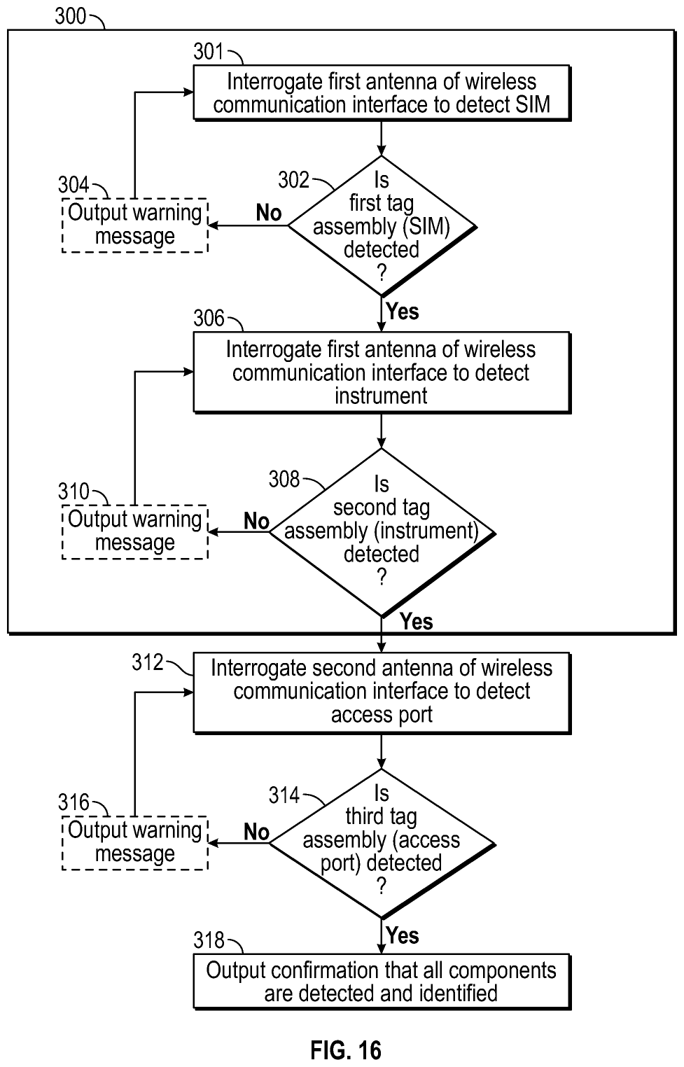

With reference to FIG. 16, a flow chart of a method for component detection and identification of the components, i.e., the SIM 80, the instrument 50, and the access port 55 are properly coupled to the IDU 52 and interconnected with each other and the holder 46, including the port latch 46c. This process may be performed at any point at which instrument 50 is attached to the IDU 52 (e.g., during initial setup of the robotic arm 40 or during replacement of the instrument 50) to verify all of the components have been properly engaged.

At step 300, the main cart controller 41a signals the wireless communication interface 100 to interrogate the first antenna 102 to determine the state of the coupling between the IDU 52, the SIM 80, and the instrument 50. The interrogation process may be initiated automatically based on the current state of the system 10, e.g., instrument setup or replacement, or manually by the user via a graphical user interface displayed on of the displays 23, 32, and/or 34. The interrogation includes establishing wireless communication between the first antenna 102 and the first tag assembly 200 at step 301. As noted above, the communication is established only if the first tag assembly 200 is completely assembled, which occurs once the SIM 80 is mechanically engaged with the IDU 52, thereby closing the electrical circuit. Thus, at step 302, the main cart controller 41a determines whether the SIM 80 is properly coupled to the IDU 52 based on whether the tag assembly 200 is responsive. If there is no response, at step 304, the main cart controller 41a may output a warning message that the SIM 80 is unresponsive, indicating to the user to check the connection between the IDU 52 and the SIM 80 and to repeat interrogation.

Once it is established that the SIM 80 is coupled to the IDU 52, the main cart controller 41a proceeds to determine whether the instrument 50 is coupled to the SIM 80. At step 306, the interrogation includes establishing wireless communication between the first antenna 102 and the second tag assembly 210. As noted above, the communication is established only if the second tag assembly 210 is completely assembled, which occurs once the instrument 50 is mechanically engaged with the SIM 80. Thus, at step 308, the main cart controller 41a determines whether the instrument 50 is properly coupled to the SIM 80 based on whether the second tag assembly 210 is responsive. If there is no response, at step 310, the main cart controller 41a may output a warning message that the instrument 50 is unresponsive, indicating to the user to check the connection between the SIM 80 and the instrument 50 and to repeat interrogation.

The process also includes verifying whether the access port 55 is engaged by the port latch 46c. The verification is performed by the wireless communication interface 100 using the second antenna 104. At step 312, the interrogation includes establishing wireless communication between the second antenna 104 and the antenna 223. At step 314, the main cart controller 41a determines whether the access port 55 is properly coupled to the port latch 46c based on whether the antenna 223 is responsive. If there is no response, at step 316, the main cart controller 41a may output a warning message that the access port 55 is unresponsive, indicating to the user to check the connection between the access port 55 is and the port latch 46c and to repeat interrogation. The access port 55 may be interrogated either before or after interrogation of the steps 300-310 using the first antenna 102. Similarly, other components may be interrogated during the process, such as surgical drapes, which may have RFID tags embedded therein. If all of the antennas 203, 213, 223 are verified, at step 318, a confirmation may be output indicating that all of the components are detected and the robotic arm 40 is ready for use.

The wireless communication interface 100 may also be used to detect collisions between robotic arms 40. In particular, the wireless communication interface 100 of one robotic arm 40 may be used to interrogate the wireless communication interfaces 100 of other robotic arms 40 and upon detection a proximity alarm may be output and/or movement of the robotic arm 40 may be stopped or temporarily interrupted. The alarm may be resolved once the robotic arms 40 are moved apart to a sufficient distance.

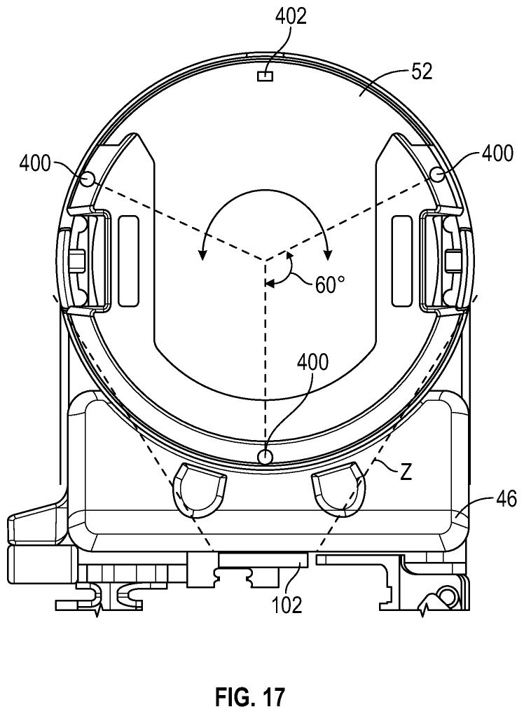

FIG. 17 shows another embodiment of RFID tags 400 disposed on the IDU 52. As described above, the holder 46 includes the wireless communication interface 100 and the first antenna 102. The first antenna 102 has a reading zone “Z” that extends toward the IDU 52 allowing for interrogation of the RFID tags 400. The reading zone “Z” may have any suitable shape, e.g., conical cross-section, that depends on the shape of the first antenna 102. The IDU 52 is configured to rotate about a longitudinal axis passing through the IDU 52 and relative to the holder 46. Rotation of the IDU 52 along with the instrument 50 allows for orientation of the end effector during the surgical procedure. However, rotation of the IDU 52 also move the RFID tags 400 out of the reading zone “Z”, which disrupts continuous communication of the RFID tags 400 with the wireless communication interface 100.

The IDU 52 includes a plurality of tags 400 that are evenly spaced along the periphery of the IDU 52, such that regardless of the rotational position of the IDU 52 relative to the first antenna 102, at least one of the RFID tags 400 is disposed within the reading zone “Z” allowing for uninterrupted communication between the RFID tag 400 and the first antenna 102. Two or more tags 400 may be used, such that two (2) RFID tags 400 are disposed at diametrically opposed points on the IDU 52, 1800 apart. Thus, the RFID tags 400 are disposed in equal, angular increments of 360°/n, e.g., when three (3) tags 400 are used, the RFID tags 400 are spaced 120°, six (6) tags 400 are used, the RFID tags 400 are spaced 60°, etc. Furthermore, the RFID tags 400 may be disposed along any longitudinal position of the IDU 52 and do not need to be placed on the same radial plane. In other words, the RFID tags 400 are disposed along longitudinal axes that are evenly spaced apart as described above.

The RFID tags 400 may be coupled to a one or more integrated chips 402 storing data (e.g., identification data), a sensor, or another device outputting data for reading by the wireless communication interface 100. Sensors may be any suitable sensor including but not limited to current sensors, torque sensors, position sensors (linear and angular), temperature sensors, and the like. The sensors may be disposed in the IDU 52 and/or the instrument 50 and are coupled to the RFID tags 400 allowing for continuous wireless transmission of sensor data regardless of rotational position of the IDU 52.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

1. A surgical robotic arm comprising:
a robotic joint;
an instrument drive unit configured to couple to the robotic joint;
a sterile interface module configured to couple to the instrument drive unit;
a first RFID tag assembly including a first portion disposed in the instrument drive unit and a second portion disposed in the sterile interface module, wherein the first portion of the first RFID tag assembly and the second portion of the first RFID tag assembly are in electrical communication once the instrument drive unit is mechanically engaged with the sterile interface module; and
a wireless communication interface configured to interrogate the first RFID tag assembly.

2. The surgical robotic arm according to claim 1, further comprising:
a latch port configured to couple to an access port.

3. The surgical robotic arm according to claim 2, wherein the wireless communication interface includes a first antenna disposed along at least a portion of the robotic joint and a second antenna disposed at the latch port.

4. The surgical robotic arm according to claim 1, wherein the instrument drive unit is configured to move longitudinally along the robotic joint.

5. The surgical robotic arm according to claim 1, wherein the first portion of the first RFID tag assembly includes a first pair of contacts, and the second portion of the first RFID tag assembly includes a second pair of contacts, the first pair of contacts and the second pair of contacts are configured to electrically couple to each other once the instrument drive unit is mechanically engaged with the sterile interface module.

6. The surgical robotic arm according to claim 1, wherein the first RFID tag assembly includes a first RFID tag antenna.

7. The surgical robotic arm according to claim 1, further comprising:
an instrument configured to couple to the sterile interface module; and
a second RFID tag assembly including a first portion disposed in the sterile interface module and a second portion disposed in the instrument, wherein the first portion of the second RFID tag assembly and the second portion of the second RFID tag assembly are in electrical communication once the instrument is mechanically engaged with the sterile interface module;
wherein the wireless communication interface is configured to interrogate the second RFID tag assembly.

8. The surgical robotic arm according to claim 7, wherein the first portion of the second RFID tag assembly includes a first pair of contacts, and the second portion of the second RFID tag assembly includes a second pair of contacts, the first pair of contacts and the second pair of contacts of the second RFID tag assembly are configured to electrically couple to each other once the instrument is mechanically engaged with the sterile interface module.

9. The surgical robotic arm according to claim 8, wherein the second RFID tag assembly includes a second RFID tag antenna.

10. A surgical robotic arm comprising:
a robotic joint;
a first component configured to couple to the robotic joint;
a second component configured to couple to the first component;
a first RFID tag assembly including a first portion disposed in the first component and a second portion disposed in the second component, wherein the first portion of the first RFID tag assembly and the second portion of the first RFID tag assembly are in electrical communication once the first component is mechanically engaged with the second component; and
a wireless communication interface configured to interrogate the first RFID tag assembly.

11. The surgical robotic arm according to claim 10, wherein the first component is an instrument drive unit.

12. The surgical robotic arm according to claim 11, wherein the second component is a sterile interface module configured to secure a sterile drape to the instrument drive unit.

13. The surgical robotic arm according to claim 10, wherein the first portion of the first RFID tag assembly includes a first pair of contacts, and the second portion of the first RFID tag assembly includes a second pair of contacts, the first pair of contacts and the second pair of contacts are configured to electrically couple to each other once the first component is mechanically engaged with the second component.

14. The surgical robotic arm according to claim 10, wherein the first RFID tag assembly includes a first RFID tag antenna.

15. The surgical robotic arm according to claim 10, further comprising:
a third component configured to couple to the second component; and
a second RFID tag assembly including a first portion disposed in the second component and a second portion disposed in the third component, wherein the first portion of the second RFID tag assembly and the second portion of the second RFID tag assembly are in electrical communication once the third component is mechanically engaged with the second component;
wherein the wireless communication interface is configured to interrogate the second RFID tag assembly.

16. The surgical robotic arm according to claim 15, wherein the first portion of the second RFID tag assembly includes a first pair of contacts, and the second portion of the second RFID tag assembly includes a second pair of contacts, the first pair of contacts and the second pair of contacts of the second RFID tag assembly are configured to electrically couple to each other once the third component is mechanically engaged with the second component.

17. The surgical robotic arm according to claim 16, wherein the second RFID tag assembly includes a second RFID tag antenna.

18. A method for detecting components of a surgical robotic arm, the method comprising:
interrogating a first RFID tag assembly including a first portion disposed in a first component and a second portion disposed in a second component, wherein the first portion of the first RFID tag assembly and the second portion of the first RFID tag assembly are in electrical communication once the first component is mechanically engaged with the second component and at least one of the first component and the second component is coupled to a robotic joint;
determining whether the first component and the second component are mechanically engaged to each other based on the interrogation; and
outputting a confirming based on the interrogation that the first component and the second component are mechanically engaged to each other.

19. The method according to claim 18, wherein interrogating is performed using a wireless communication interface disposed in the robotic joint.

20. The method according to claim 19, further comprising:
interrogating a second RFID tag assembly including a first portion disposed in the second component and a second portion disposed in a third component, wherein the first portion of the second RFID tag assembly and the second portion of the second RFID tag assembly are in electrical communication once the third component is mechanically engaged with the second component.

21-27. (canceled)
